(19) United States
(12) Patent Application Publication
(10) Pub. No.: US 2010/0216195 A1
(43) Pub. Date: Aug. 26, 2010

(54) Enzymatic Production of Sucrose-6-Ester, an Intermediate for the Manufacturing of Halo Sugars...

(76) Inventors: Rakesh Ratnam, Karnataka (IN); Batchu Chandrashekar, Karnataka (IN); Pampapathy Subramaniyam, Karnataka (IN)

Correspondence Address: THE NATH LAW GROUP, 112 South West Street, Alexandria, VA, 22314, US

(21) Appl. No.: 12/086,175

(22) PCT Filed: Nov. 28, 2006
(86) PCT No.: PCT/IN2006/000478

§ 371 Date: Aug. 7, 2008

(30) Foreign Application Priority Data
Dec. 9, 2005 (IN) 1522/MUM/2005

(51) Int. Cl. C12P 19/12

(52) U.S. Cl. 435/100

(57) ABSTRACT

A novel process is described for production of 6-acyl-sucrose comprising enzymatic acylation of sucrose by an esterifying agent including an organic acid in presence of a lipase or an esterase in a solvent in which the enzyme used is stable. Chlorinated sucrose, the high intensity sweetener trichlorogalactosucrose can be prepared by chlorination and deacylation of 6-acyl sucrose prepared by the process of this invention.

### TECHNICAL FIELD

The present invention relates to enzymatic production of sucrose-6-ester, an intermediate used in production of halo (chlorinated) sugars including 1′-6′-Dichloro-1′-6′-DIDEOXY-β-Fructofuranasyl-4-chloro-4-deoxy-galactopyranoside (TGS) and its precursor (TGS-6-ester).

### BACKGROUND OF THE INVENTION

Strategies of prior art methods of production of 4,1′, 6′ trichlorogalactosucrose (TGS) predominantly involve chlorination of sucrose-6-ester by use of Vilsmeier-Haack reagent derived from various chlorinating agents such as phosphorus oxychloride, oxalyl chloride, phosphorus pentachloride etc, and a tertiary amide such as dimethyl formamide (DMF) or dimethyl acetamide to chlorinate Sucrose-6-ester, to form 6 acetyl 4,1′, 6′ trichlorogalactosucrose. After the said chlorination reaction, the reaction mass is neutralized to pH 7.0-7.5 using appropriate alkali hydroxides of calcium, sodium, etc. and then pH preferably increased still further to deesterify/deacetylate the 6 acetyl 4,1′, 6′ trichlorogalactosucrose to form 4,1′, 6′ trichlorogalactosucrose (TGS).

Sucrose-6-ester is usually derived by esterification of sucrose, is a precursor of TGS—a zero calorie high intensity sweetener or taste modifier used in food and other applications. However, the esterification of sucrose has to be carried out at the 6^{th} position alone and this is a major challenge for its manufacture because the position at which this esterification is aimed at is lesser reactive than other more reactive competing positions i.e. 1′ and 6′ positions

To achieve regioselective esterification, various methods have been described in the organic synthesis way of manufacture of sucrose-6-ester including but not limited to by tin mediated adduct formation followed by esterification and direct esterification of the sucrose in pyridine. However, methods via organic synthesis, even the regioselctive ones, result in formation of various by products and isolation procedures have to be evolved to purify the sucrose-6-ester prior to chlorination. Further improvement is required in achieving more control on site-specific esterification.

### SUMMARY OF THE INVENTION

The invention discloses a process of enzymatic acylation wherein a 6-acyl sucrose is major product when sucrose is reacted with a suitable acyl or aryl esterifying agent, including an organic acid, in presence of a novel lipase enzyme or cross linked lipase enzyme either in free or immobilized form in the presence or absence of the tertiary amide or in any other suitable solvent in which the enzyme is stable. The ester group introduced into the 6^{th} position of sucrose molecule could be an alkyl, aryl, substituted alkyl or substituted aryl group which depends on the reactant used for the esterification. The 6-acyl-sucrose thus obtained can be used for preparation of halo sugars.

### PRIOR ART

Dordick et al (1992) in U.S. Pat. No. 5,128,248, have disclosed a process for acylating sucrose or a derivative thereof on at least one of the 4′- and 6-positions, in which specifically a donor acyl ester is reacted with sucrose or a derivative thereof in a non-hydroxylic solvent in the presence of a microbial lipase. The said donor ester is a reactive ester of an alkanoic acid or benzoic acid.

Bornemann et al (1992) in U.S. Pat. No. 5,141,860, have disclosed a method for the preparation of partly deacylated acylate of sucrose having acyl groups at least at the 2-, 3-, and 3′-positions and at least one free hydroxyl group in each ring, in which a sucrose octaacylate is treated with an enzyme or combination of enzymes capable of catalyzing the hydrolysis of at least one acyl group from each ring of said sucrose octaacylate in an aqueous medium comprising water and up to 50% organic solvent buffered to a pH of 5-7, and isolating the resulting partly deacylated sucrose acylate, said enzymes being selected from the group consisting of pancreatic lipases, yeast esterase, fungal .alpha.-amylases, subtilisins, Aspergillus melleus protease and .alpha.-galactosidases

### DETAILED DESCRIPTION OF THE INVENTION

Enzymatic routes are far more specific in their end products. They are very substrate specific too.

This invention describes a novel way of producing sucrose-6-ester by use of enzymes. A highly efficient and selective enzymatic esterification of sucrose is described. The regioselective reaction is carried out by a novel lipase enzyme or cross linked lipase enzyme either in free or immobilized form in the presence or absence of the tertiary amide or in any other suitable solvent in which the enzyme is stable. The ester group introduced into the 6^{th} position of sucrose molecule could be an alkyl, aryl, substituted alkyl or substituted aryl group which depends on the reactant used for the acylation. The 6-acyl-sucrose thus obtained can be used for preparation of halo sugars such as TGS, which are used as high intensity sweetener.

The enzymes used could be esterases, lipases, etc. These enzymes can be immobilized in or on synthetic polymeric supports such as, but not limited to polyacrylic, or polystyrene or polyacrylamide, nylon based supports; or semisynthetic or natural organic supports like those based on polysaccharides such as, but not limited to cellulose, starch, dextran, agarose, chitosan, chitin, etc.; or inorganic supports like those based on carbon, silica, zirconia, alumina, zirconium phosphate, etc.

The source of the enzyme lipase can be of animal, plant or microbial origin, more preferably microbial or bacterial origin such as Bacillus thermocatenulatusis, Pseudomonas aeruginosa, etc., fungal origin such as Penicillium Roquefortii, Asperigillus niger, Asperigillus oryzae, Rhizopus niveus, Candida rugosa, Rhizomucor miheii, Candida antartctica, etc. or equivalent.

This strategy, in effect enhances the yield and purity of sucrose-6-ester, which is taken for the chlorination step as such or after the removal of solvents, for the preparation of Chlorosucrose derivatives, which in its turn improves the purity and yield of Chlorinated sucrose produced.

In this invention the enzymatic conversion of sucrose to sucrose-6-acetate essentially involves the use of sucrose and acetic acid or a suitable organic acid or a suitable acyl or aryl esterifying agent—as the reactants to directly produce sucrose-6-ester as a major product

The following invented process is a highly efficient regioselective reaction wherein for the first time, selective esterification of sucrose is carried out exclusively at the 6^{th} position by a novel isolated lipase enzyme.

In this invented process, this reaction is carried out by dissolving sucrose in moisture free DMF and was treated with the lipase enzyme. The sucrose concentration in DMF solution varies from 1:1 to 1:10 w/v. Acetic acid is used as an acylating agent and is directly added to the reaction mixture. Any other aliphatic acid, substituted aliphatic acid, aromatic acid or substituted aromatic acid can be used to produce the respective sucrose-6-ester. The temperature during the reaction can be anywhere between 15° C. to 60° C. The enzymatic esterification is completed with generation of negligible amounts of by products if any over a period between 1 hour to 16 hours. The conversion of sucrose to sucrose-6-ester is appreciably good and specific for 6^{th} position only with appropriate maintenance of reaction conditions. The enzyme can be used either in free form as powder or liquid and also in immobilized form.

The enzyme is recovered when used in immobilized form. The immobilized enzyme can be packed in a column and passing the said reactants at a set flow rate to carry out reaction. Alternatively, the reaction is carried out with the immobilized enzyme in a reactor and after the reaction, the enzyme can be recovered by filtering it off from the reaction mass.

The sucrose-6-ester thus obtained is substantially pure and is easily isolated and taken for chlorination for the production of halo sugars.

Described in the following are examples, which illustrate working of this invention without limiting the scope of this invention in any manner. Reactants, proportion of reactants used, range of reaction conditions described are only illustrative and the scope of this invention extends to their analogous reactants, reaction conditions and reactions of analogous generic nature. In general, any equivalent alternative, which is obvious to a person skilled in art of chlorinated sucrose production is covered within the scope of this specification. Mention in singular is construed to cover its plural also, including all equivalent alternatives encompassed by that expression, unless the context does not permit so, viz: use of “a chlorinated sucrose” includes all chlorinated sucrose compounds individually as well as mixtures thereof or an alternative chlorinated sucrose compound that may perform same function in a relevant context. A mention of “an organic solvent” for solution covers use of one or more of an organic solvent in succession or in a combination as a mixture or any one of the several alternatives capable of performing same function as claimed, described in the description or illustrated in one or more of an example. In this specification, sucrose-6-ester and 6-acyl-sucrose have been used interchangeably as equivalents to each other for all functional purposes.

### Example 1

### Enzymatic Acetylation of Sucrose in DMF

Lipase from Asperigillus oryzae was immobilized on Polystyrene beads and cross linked with glutaraldehyde to get immobilized lipase. 200 g of sucrose was dissolved in 800 ml of DMF at 80° C. and was cooled to room temperature, 34 g of the said immobilized lipase was added and was kept stirring in a reaction flask. The temperature was maintained at 30° C. 13.5 g of acetic acid was added dropwise to the reaction flask with constant stirring. The stirring was continued and the acetylation was monitored by TLC and HPLC.

Acetylation up to 70% was achieved within 3 hours and the reaction contents were filtered and the enzyme was washed with water and recovered.

The sucrose-6-acetate formation was 70% with no by products produced as confirmed by HPLC.

### Example 2

### Enzymatic Acetylation of Sucrose in Isoamyl Alcohol

20 g of sucrose was partially dissolved in 400 ml of Isoamyl alcohol at 80° C. and was cooled to room temperature. 34 g of immobilized lipase enzyme from Asperigillus oryzae, as prepared by process described in Example 1, was added and was kept stirring in a reaction flask. The temperature was maintained at 30° C. 3.5 g of acetic acid was added dropwise to the reaction flask with constant stirring. The stirring was continued and the acetylation was monitored by TLC and HPLC.

Acetylation up to 70% was achieved within 3 hours and the reaction contents were filtered and the enzyme was washed with water and recovered. The sucrose-6-acetate formation was 70% with no by products produced as confirmed by HPLC.

### Example 3

### Enzymatic Acylation of Sucrose in DMF Using Benzoic Anhydride

10 g of sucrose was dissolved in 100 ml of DMF at 50° C. and was cooled to 25° C. 26 g of lipase enzyme isolated from Pseudomonas sp. was added and was stirred thoroughly. The temperature was again raised to 50° C. 0.59 ml of Benzoic anhydride was added and the reaction was continued for 6.0 hours. The acylation was monitored by TLC as well as HPLC.

Benzoylation was achieved up to 48% in 6 hours with no by product formation.

### Example 4

### Enzymatic Acylation of Sucrose in DMSO Using Lauric Acid

10 g of sucrose was dissolved in 100 ml of DMSO (Dimethyl Sulphoxide) at 60° C. and was cooled to 25° C. 26 g of lipase enzyme isolated from Rhizopus sp. was added and was stirred thoroughly. The temperature was again raised to 50° C. 11.69 g of Lauric acid was added and the reaction was continued for 8.0 hours. The acylation was monitored by TLC as well as HPLC.

Acylation was achieved up to 42% in 8 hours with no by product formation as confirmed by HPLC.

### Example 5

### Enzymatic Acylation of Sucrose in DMSO Using P-Nitro Benzoic Acid

10 g of sucrose was dissolved in 100 ml of DMSO at 60° C. and was maintained at 35° C. 26 g of lipase enzyme isolated from pseudomonas sp. was added and was stirred thoroughly. The temperature was again raised to 60° C. 4.89 g of p-nitro benzoic acid was added and the reaction was continued for 8.0 hours. The benzoylation was monitored by TLC as well as HPLC.

Benzoylation was achieved up to 32% in 8 hours with no by product formation as confirmed by HPLC.

### Example 6

### Enzymatic Acetylation and Chlorination for the Preparation of TGS

In one experiment, 200 g of sucrose was dissolved in 2000 ml of DMF at 80° C. and was cooled to room temperature. 34 g of immobilized lipase enzyme from Asperigillus oryzae, prepared by a process described in Example 1, was added and was kept stirring in a reaction flask. The temperature was maintained at 50° C. 13.8 ml of acetic anhydride was added dropwise to the reaction flask with constant stirring. The stirring was continued and the acetylation was monitored by TLC and HPLC.

Acetylation up to 68% was achieved within 6 hours and the reaction contents were filtered and the enzyme was washed with water and recovered. The DMF solution was then taken for chlorination.

432 g of PCl₅ was added to 2 L of DMF at 35° C. and the Vilsmeier Haack reagent was allowed to form. The POCl₃ generated from the reaction formed the second Vilsmeier with the available DMF in the reaction mass and the reaction mass was stirred thoroughly for 60 minutes. The reaction mass was then cooled to 0° C. and the 6-acyl sucrose in DMF obtained from the enzymatic reaction was added slowly under stirring. After the addition of the 6-acyl sucrose, the reaction mass was heated to 35° C. and was maintained under stirring for 60 minutes. Then the reaction mass was heated to 85° C., maintained for 60 minutes, again heated to 100° C., maintained for 6 hours and then further heated to 114° C. and maintained for 1.5 hours and then cooled to 65° C.

The reaction mass was then neutralized using calcium hydroxide slurry in water up to pH 7.0 and then filtered. The filtrate was then extracted into 1:3 times v/v of ethyl acetate and was concentrated to 50% of its original volume. The extract was then washed with 1:0.1 times v/v of saturated sodium chloride solution. The sodium chloride washing was repeated 12 times and the DMF content of the ethyl acetate extract was reduced to <0.1%. The ethyl acetate was then completely removed and the syrup was subjected to chromatography on silanized silica gel. The mobile phase used was a buffer solution at pH 10.5-11.0.

The pure fractions obtained from chromatographic purification was pooled together and then the pH was adjusted to 9.0 using sodium hydroxide solution. The deacetylation was allowed to complete and was confirmed by TLC.

After deacetylation, the fractions were concentrated by molecular separation using RO membrane. The concentrate after RO concentration was extracted into 1:3.5 times v/v of ethyl acetate and the layers were separated. The ethyl acetate extract was concentrated to maximum and the crystals obtained were re-dissolved in methanol. The methanol solution was then filtered to remove any extraneous materials and was concentrated and crystallized.

The purity obtained was 98.5% by HPLC and the overall yield obtained from 6-acyl sucrose input was found to be 35%.

### Example 7

### Enzymatic Phthalation Using Esterase in T-Butanol

25 g of sucrose was partially dissolved in 100 ml of t-butanol at 60° C. and was cooled to 25° C. 45 g of esterase isolated from candida sp. was added and was stirred thoroughly. The temperature was again raised to 60° C. 4.89 g of phthallic acid was added and the reaction was continued for 16.0 hours. The phthalation was monitored by TLC as well as HPLC.

Phthalation was achieved up to 26% in 16 hours with no by-product formation as confirmed by HPLC.

### Example 8

### Enzymatic Acylation Using Immobilized Lipase Packed in Column

25 g of sucrose was partially dissolved in 100 ml of DMF at 80° C. and was cooled to 25° C. 15 g of immobilized lipase on Polystyrene support from Pseudomonas sp was packed in a glass column. The inlet of the column was connected to the sucrose solution in DMF through a peristaltic pump. The outlet was also connected to the sucrose solution. The solution was kept stirring at 25° C. 4.0 ml of acetic acid was added to the sucrose solution and was pumped into the glass column through the peristaltic pump at a flow rate of 20 ml per hour. This re-circulation was continued for 12 hours. The Acetylation reaction was monitored by TLC periodically.

Acetylation was achieved up to 59% in 12 hours with no by-product formation as confirmed by HPLC.

1. A process for acylating sucrose predominantly on 6-position to prepare 6-acyl-sucrose, in which an enzyme is used which is capable of catalyzing selective acylation at 6^{th} position of sucrose molecule when organic acid, comprising an alkanoic acid or aryl carboxylic acid, or an acylating agent is reacted with sucrose in a solvent; the said solvent is a solvent in which the said enzyme is stable.

2. A process of claim 1 wherein:
a. the said organic acid comprising alkanoic acid or aryl carboxylic acid further comprises acetic acid, propionic acid, butyric acid, hexaenoic acid, benzoic acid, phthallic acid and the like,
b. the said acylating agent comprises acetic anhydride, propionic anhydride, lauric anhydride, butyric anhydride, benzoic anhydride, phthallic anhydride and the like,
c. the said enzyme comprises a lipase or an esterase, in a soluble or an immobilized form and derived from an animal, plant or a microorganism,
d. the said solvent in which the said enzyme is stable comprises Dimethylformamide (DMF), Isoamyl alcohol, Octanol, Hexane, Cyclohexane, Toluene, t-butanol, dimethyl sulphoxide and the like.

3. A process of claim 1 comprising following steps:
a. sucrose is dissolved in a solvent to produce a solution, preferably in a moisture free solvent and the said solvent being the one in which the said enzyme is stable,
b. lipase or esterase is added to the said solution,
c. to the reaction mixture of preceding step is added an acetic acid, or an another organic acid, or an acylating agent,
d. the reaction is allowed to proceed at a temperature which facilitates the enzyme action preferably between 15° to 60° celcius, for a period of time enough to get practically maximum conversion of sucrose to 6-acyl-sucrose preferably for about 1 to 16 hours.

4. A process of claim 1 of acylation of sucrose comprising a reactor in which the said enzyme in an immobilized form contacts with a recirculating solution containing sucrose and an organic acid or an acylating agent, at a temperature and for a period of time sufficient to acylate major quantity of sucrose into 6-acyl-sucrose.

5. A process of claim 4 wherein:
a. sucrose is dissolved in a solvent, preferably partially dissolved in DMF, at a temperature preferably of around 80° Celcius and was cooled to a temperature preferably of around 25° Celcius,
b. a preferred enzyme lipase extracted from pseudomonas sp immobilized on a preferred Polystyrene support is packed in a glass column,
c. inlet of the column is connected to the sucrose solution in DMF through a pump, preferably a peristaltic pump,
d. the outlet is connected to the said sucrose solution referred in sub-claim (a.) of this claim,
e. the solution is kept stirring preferably at around 25° C.,
f. an organic acid, preferably acetic acid is added to the sucrose solution and pumped into the glass column through the peristaltic pump at a flow rate preferably of about 20 ml per hour, the re-circulation continued for a period of time, preferably around 12 hours, to get conversion of a significant portion of sucrose into 6-acyl-sucrose,
g. and optional use of the 6-acyl-sucrose solution thus obtained to prepare chlorinated sucrose.

6. A process of claim 1 wherein the resulting process stream containing 6-acyl-sucrose is subjected to chlorination and deacetylation resulting into production of a chlorinated sucrose including the high-intensity sweetener 4,1′, 6′ trichlorogalactosucrose (TGS).
